(19) United States
(12) Patent Application Publication
(10) Pub. No.: US 2026/0069256 A1
(43) Pub. Date: Mar. 12, 2026

(54) MEDICAL DRAIN BAG AND IMAGING SLIDE

(71) Applicant: Honeywell International Inc., Charlotte, NC (US)

(72) Inventor: Daniel James YEE, Fort Mill, SC (US)

(21) Appl. No.: 18/883,909

(22) Filed: Sep. 12, 2024

(51) Int. Cl. A61B 10/00; A61M 1/14; B01L 3/00

(52) U.S. Cl. CPC A61B 10/0045; A61M 1/159; B01L 3/5088; A61B 2010/0061; B01L 2300/0822; B01L 2300/168

(57) ABSTRACT

A medical drain bag includes a main body that defines a reservoir. The main body is configured to hold a liquid within the reservoir. The medical drain bag includes a plurality of ports that are each in fluid communication with the reservoir. The plurality of ports include an input port configured to be coupled to a catheter and receive the liquid through the catheter, a syringe port that is configured to allow the liquid to flow from the reservoir and to a syringe, and a sample port that is coupled to an imaging slide and is configured to allow the liquid to flow from the reservoir and to the imaging slide. The imaging slide includes a channel configured to receive the liquid, an imaging window that at least partially defines the channel, and an inlet that is coupled to the sample port.

### TECHNICAL FIELD

Example embodiments of the present disclosure relate generally to a medical drain bag for receiving a liquid and an imaging slide for imaging the liquid. The liquid within the imaging slide can subsequently be imaged by an imaging system, such as a digital microscope.

### BACKGROUND

Fluid samples from patients (e.g., blood, urine, or peritoneal dialysis effluent) are often analyzed for identification and classification of white blood cells to determine whether a patient has an infection. Conventional methods of collecting the fluid samples for analysis are often time consuming or difficult when the fluid sample is being collected and imaged by the patient or an individual that is unskilled or not a medical professional.

For example, peritoneal dialysis (PD) is often performed outside of a clinic and at a patient’s home. It may be beneficial to collect the image data of the PD effluent within a relatively short time frame from when the fluid flows out of the patient’s body. For example, it may be more beneficial for the image data of the PD effluent to be generated at the patient’s home instead of delivering the fluid sample to a lab or a clinic for analysis. As such, an easier method of collecting the fluid sample for analysis would be welcomed in the art.

Through applied effort, ingenuity, and innovation, many of these identified deficiencies and problems of conventional methods have been solved by developing solutions that are structured in accordance with the embodiments of the present disclosure, many examples of which are described in detail herein.

### BRIEF SUMMARY

In general, embodiments of the present disclosure provided herein include apparatuses to provide for an improved medical drain bag and imaging slide.

In various aspects a medical drain bag includes a main body that defines a reservoir, the main body configured to hold a liquid within the reservoir; and a plurality of ports that are each in fluid communication with the reservoir, wherein the plurality of ports include: an input port configured to be coupled to a catheter and receive the liquid through the catheter; a syringe port that is configured to allow the liquid to flow from the reservoir and to a syringe; and a sample port that is configured to allow the liquid to flow from the reservoir and to an imaging slide.

In various examples, the medical drain bag includes a plurality of tubes that are each coupled to the main body, wherein the plurality of tubes include: an input tube that is coupled to the input port; a syringe tube that is coupled to the syringe port; and a sample tube that is coupled to the sample port.

In various examples, the main body has a rounded rectangle shape that defines four edges, and wherein each of the plurality of tubes are coupled proximate to the same edge of the main body.

In various examples, the main body has a rounded rectangle shape that defines a first edge that is opposite to a second edge, the input tube and the syringe tube are coupled proximate to the first edge of the main body, and the sample tube is coupled proximate to the second edge of the main body.

In various examples, the techniques described herein relate to a medical drain bag, further including a safety clamp coupled to the input tube, the syringe tube, or the sample tube.

In various examples, the liquid is a peritoneal dialysis effluent.

In various aspects, an imaging slide configured to receive a flow of a liquid includes a channel configured to receive the flow of the liquid; an imaging window that at least partially defines the channel; and an inlet, wherein the inlet is configured to allow a catheter or a sample tube of a medical drain bag to be coupled to the imaging slide.

In various examples, the imaging window is optically clear, wherein the imaging slide is configured to be imaged by an imaging system, wherein the imaging system includes an illumination device and an imaging device, and wherein the imaging system is configured to receive the imaging slide between the illumination device and the imaging device.

In various examples, the imaging slide includes a protective film that is placed over the imaging window.

In various examples, wherein the protective film includes a tab that extends beyond a periphery of a main body of the imaging slide.

In various examples, the protective film wraps around an edge of a main body of the imaging slide such that the protective film is placed over opposing sides of the imaging slide.

In various aspects, a medical drain bag includes a main body that defines a reservoir, the main body configured to hold a liquid within the reservoir; and a plurality of ports that are each in fluid communication with the reservoir, wherein the plurality of ports include: an input port configured to be coupled to a catheter and receive the liquid through the catheter; a syringe port that is configured to allow the liquid to flow from the reservoir and to a syringe; and a sample port that is coupled to an imaging slide and is configured to allow the liquid to flow from the reservoir and to the imaging slide, wherein the imaging slide includes: a channel configured to receive the liquid; an imaging window that at least partially defines the channel; and an inlet, wherein the inlet is coupled to the sample port.

In various examples, the medical drain bag includes a plurality of tubes that are each coupled to the main body, wherein the plurality of tubes include: an input tube that is coupled to the input port; a syringe tube that is coupled to the syringe port; and a sample tube that is coupled to the sample port.

In various examples, the main body has a rounded rectangle shape that defines four edges, and wherein each of the plurality of tubes are coupled proximate to the same edge of the main body.

In various examples, the main body has a rounded rectangle shape that defines a first edge that is opposite to a second edge, the input tube and the syringe tube are coupled proximate to the first edge of the main body, and the sample tube is coupled proximate to the second edge of the main body.

In various examples, the medical drain bag includes a safety clamp coupled to the input tube, the syringe tube, or the sample tube.

In various examples, the imaging window is optically clear, wherein the imaging slide is configured to be imaged by an imaging system, wherein the imaging system includes an illumination device and an imaging device, and wherein the imaging system is configured to receive the imaging slide between the illumination device and the imaging device.

In various examples, the medical drain bag includes a protective film that is placed over the imaging window.

In various examples, the protective film includes a tab that extends beyond a periphery of a main body of the imaging slide.

In various examples, the protective film wraps around an edge of a main body of the imaging slide such that the protective film is placed over opposing sides of the imaging slide.

The above summary is provided merely for purposes of summarizing some example embodiments to provide a basic understanding of some aspects of the present disclosure. Accordingly, it will be appreciated that the above-described embodiments are merely examples and should not be construed to narrow the scope or spirit of the present disclosure in any way. It will be appreciated that the scope of the present disclosure encompasses many potential embodiments in addition to those here summarized, some of which will be further described below. Other features, aspects, and advantages of the subject matter will become apparent from the description, the drawings, and the claims.

### BRIEF DESCRIPTION OF THE DRAWINGS

Having thus described certain example embodiments of the present disclosure in general terms above, non-limiting and non-exhaustive embodiments of the subject disclosure are described with reference to the following figures, which are not necessarily drawn to scale and wherein like reference numerals refer to like parts throughout the various views unless otherwise specified. The components illustrated in the figures may or may not be present in certain embodiments described herein. Some embodiments may include fewer (or more) components than those shown in the figures.

FIG. 1 provides a schematic depiction of a patient undergoing a peritoneal dialysis procedure, in accordance with an example embodiment.

FIG. 2 provides a front view of a medical drain bag, in accordance with an example embodiment.

FIG. 3 provides a front view of a medical drain bag, in accordance with an example embodiment.

FIG. 4 provides a front view of a medical drain bag, in accordance with an example embodiment.

FIG. 5A provides a front view of an imaging slide, in accordance with an example embodiment.

FIG. 5B provides a side view of the imaging slide of FIG. 5A, in accordance with an example embodiment.

FIG. 6 provides an isometric view of an imaging slide, in accordance with an example embodiment.

FIG. 7 provides an isometric view of the imaging slide of FIG. 6 with a protective film removed, in accordance with an example embodiment.

FIG. 8 provides an isometric view of an imaging system, in accordance with an example embodiment.

### DETAILED DESCRIPTION

One or more embodiments are now more fully described with reference to the accompanying drawings, wherein like reference numerals are used to refer to like elements throughout and in which some, but not all embodiments of the inventions are shown. In the following description, for purposes of explanation, numerous specific details are set forth in order to provide a thorough understanding of the various embodiments. It is evident, however, that the various embodiments can be practiced without these specific details. It should be understood that some, but not all embodiments are shown and described herein. Indeed, the embodiments may be embodied in many different forms, and accordingly this disclosure should not be construed as limited to the embodiments set forth herein. Rather, these embodiments are provided so that this disclosure will satisfy applicable legal requirements.

As used herein, the term “exemplary” means serving as an example, instance, or illustration. Any aspect or design described herein as “exemplary” is not necessarily to be construed as preferred or advantageous over other aspects or designs. Rather, use of the word exemplary is intended to present concepts in a concrete fashion. In addition, while a particular feature may be disclosed with respect to only one of several implementations, such feature may be combined with one or more other features of the other implementations as may be desired and advantageous for any given or particular application. Furthermore, to the extent that the terms “includes” and “including” and variants thereof are used in either the detailed description or the claims, these terms are intended to be inclusive in a manner similar to the term “comprising.”

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As used herein, the terms “upstream” and “downstream” refer to the relative direction with respect to fluid flow in a fluid pathway. For example, “upstream” refers to the direction from which the fluid flows, and “downstream” refers to the direction to which the fluid flows. As used herein, the term “fluid communication” means that a fluid is capable of making the connection between the areas specified.

As used herein, the terms “coupled,” “fixed,” “attached to,” and the like refer to both direct coupling, fixing, or attaching, as well as indirect coupling, fixing, or attaching through one or more intermediate components or features, unless otherwise specified herein.

As used herein, the term “positioned directly on” or “positioned on” refers to a first component being positioned on a second component such that they make contact. Similarly, as used herein, the term “positioned directly between” refers to a first component being positioned between a second component and a third component such that the first component makes contact with both the second component and the third component. In contrast, a first component that is “positioned between” a second component and a third component may or may not have contact with the second component and the third component. Additionally, a first component that is “positioned between” a second component and a third component is positioned such that there may be other intervening components between the second component and the third component other than the first component.

As used herein, terms such as “front,” “rear,” “top,” etc. are used for explanatory purposes in the examples provided below to describe the relative position of certain components or portions of components. Furthermore, as would be evident to one of ordinary skill in the art in light of the present disclosure, the terms “substantially” and “approximately” indicate that the referenced element or associated description is accurate to within applicable engineering tolerances.

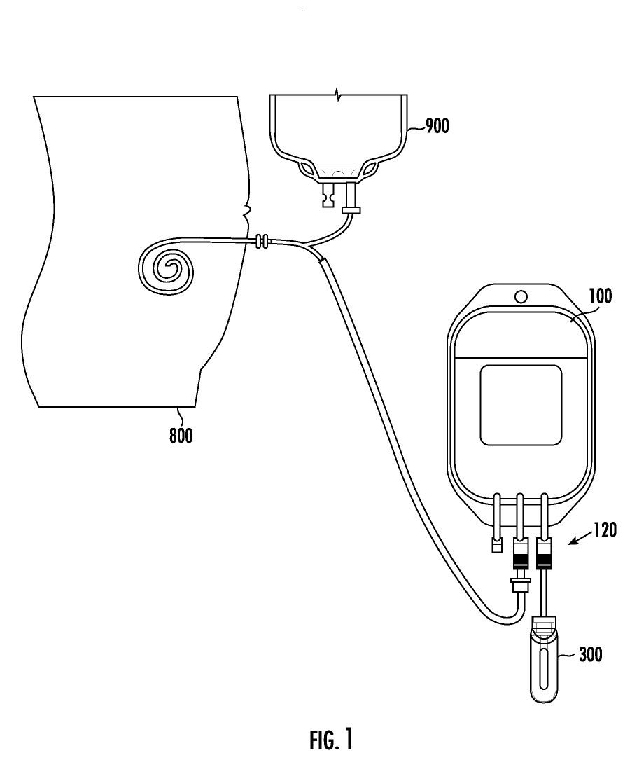

Referring now to FIG. 1, a patient 800 undergoing a peritoneal dialysis (PD) procedure is schematically depicted, according to an example embodiment. The PD procedure provides a mechanism to remove waste products from a patient’s blood when the patient’s kidneys cannot adequately function. During an example PD procedure, a cleansing fluid (such as, but not limited to, a dialysis solution such as water with sugar and other additives) flows from a treatment solution bag 900, through a tube or a pipe (such as, but not limited to, a catheter), and into the patient’s body 800. More specifically, the cleansing fluid is injected into a part of a patient’s abdomen. When the cleansing fluid is inside the patient’s body 800, the cleansing fluid absorbs waste products from the patient’s body 800. The lining of the abdomen (also known as peritoneum) can act as a filter and remove waste products from the patient’s blood. After a set period of time, the fluid with the filtered waste products (referred herein as peritoneal dialysis (PD) effluent) flows out of the patient’s abdomen, through a tube or a pipe (such as, but not limited to, a catheter), and into a medical drain bag 100.

PD procedures may be faced with some drawbacks. One of the drawbacks is that patients who undergo PD may develop infections. As such, early detection of infections after a patient undergoes PD can be beneficial for alerting patients, as well as care providers, so that early action can be taken to limit the severity and frequency of infections. Various embodiments of the present disclosures may enable such early detection of infections while a patient undergoes PD.

As will be discussed further, various embodiments of the present disclosure provide a medical drain bag 100 that provides a mechanism to collect a liquid, such as PD effluent, that is drained from a patient’s body 800. Also, and as will be discussed further, various embodiments of the present disclosure provide an imaging slide 300 for collecting and analyzing a sample of the liquid from the medical drain bag 100. The sample of the liquid within the imaging slide 300 may analyzed by an imaging system 500 (FIG. 8), which may be a point-of-care imaging system.

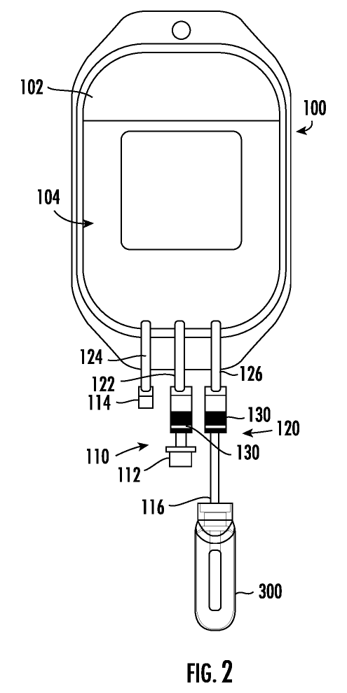

Referring now to FIGS. 2-4, front views of medical drain bags 100 are provided, in accordance with three different example embodiments. The medical drain bag 100 may include a main body 102 that defines a reservoir 104. The main body 102 may be configured to hold a liquid, such as a PD effluent, within the reservoir 104. The reservoir 104 may hold 1 liter to 5 liters of liquid. The main body 102 of the drain bag 100 may comprise a medical-grade material such as ethylene-vinyl acetate (EVA), polyethylene (PE), or ethylene vinyl alcohol (EVOH).

In various examples, the medical drain bag 100 includes a plurality of tubes 120 that are each coupled to the main body 102 of the drain bag 100. The plurality of tubes 120 may include an input tube 122, a syringe tube 124, and a sample tube 126. The medical drain bag 100 may include a plurality of ports 110. At least one of the plurality of ports 110 may include a safety cap 118 to prevent the liquid from escaping the port. The plurality of ports 110 may include an input port 112, a syringe port 114, and a sample port 116. Each of the plurality of ports 110 may be in fluid communication with the reservoir 104. For example, the input port 112 may be coupled to the input tube 122 and fluid may be allowed to flow from the reservoir 104, through the input tube 122 and to the input port 112. The syringe port 114 may be coupled to the syringe tube 124 and fluid may be allowed to flow from the reservoir 104, through the syringe tube 124 and to the syringe port 114. The sample port 116 may be coupled to the sample tube 126 and fluid may be allowed to flow from the reservoir 104, through the sample tube 126 and to the sample port 116.

In various examples, the input port 112 is configured to be coupled to a catheter and receive the liquid from the catheter. For example, and as discussed, a liquid, such as a PD effluent, may flow from a patient’s body 800 through a tube or a pipe, such as a catheter. The catheter may include an output port (not depicted). The input port 112 may be configured to mate with the output port of the catheter, which may allow the liquid to flow from the patient’s body, out the output port of the catheter, through the input port 112 of the medical drain bag 100, and into the reservoir 104 of the medical drain bag 100.

In various examples, the syringe port 114 is configured to allow the liquid to flow from the reservoir 104 and to a syringe. For example, the syringe port 114 may be configured as a needleless sample site that may allow a syringe, without a needle, to attached and draw out a sample of the liquid from the reservoir 104. The syringe port 114 may be configured as a needle sample site, which allows a needle of a syringe to be inserted into the syringe port 114 to draw out a sample of the liquid from the reservoir 104.

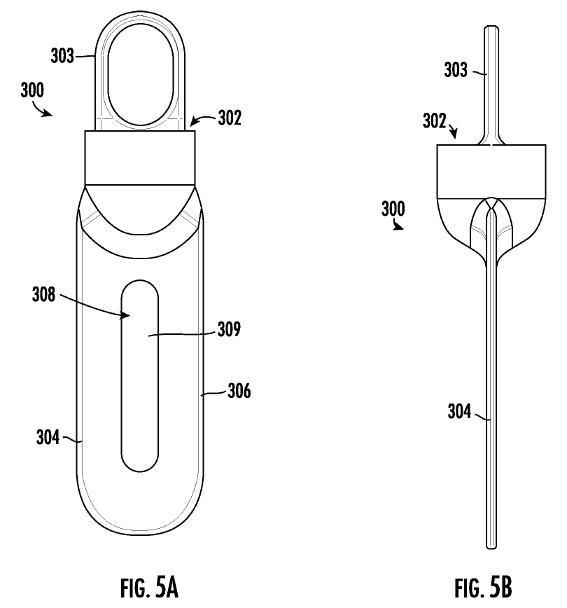

In various examples, the sample port 116 is configured to allow the liquid to flow from the reservoir 104 and to an imaging slide 300. For example, the sample port 116 may be configured to be friction fit into an inlet 302 (FIGS. 5A-7) of an imaging slide 300, which may form a seal. As another example, the sample port 116 may have threads that are configured to mesh with threads of the inlet 302 of the imaging slide 300, which may form a seal.

In various examples, the imaging slide 300 includes a safety cap 303 (FIGS. 5A-7) that would need to be removed prior to coupling the sample port 116 of the medical drain bag 100 with the inlet 302 of the imaging slide 300. The safety cap 303 may be similarly-shaped as the sample port 116 and may be configured to be friction fit into the inlet 302 of the imaging slide 300 or may have threads that are configured to mesh with threads of the inlet 302 of the imaging slide 300.

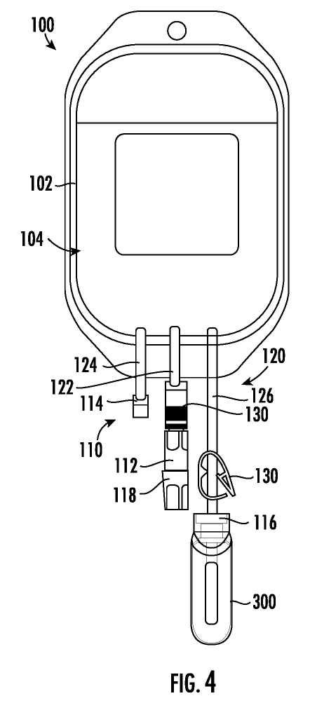

In various examples, the main body 102 of the medical drain bag 100 may have a rounded rectangle shape that defines four edges. Each of the plurality of tubes 120 may be coupled proximate to (e.g., within two inches of) the same edge of the main body 102, as depicted in FIGS. 2 and 4.

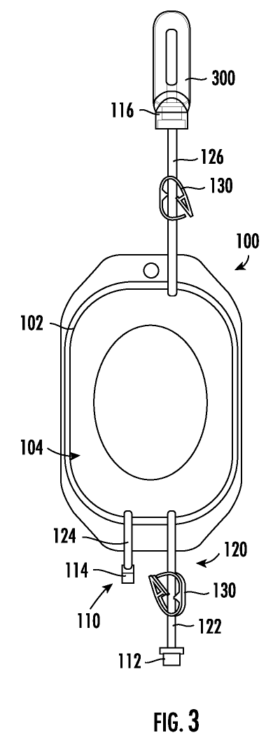

In various examples, the four edges of the medical drain bag 100 may include a first edge, such as a bottom edge, that is opposite to a second edge, such as a top edge. The input tube 122 and the syringe tube 124 may be coupled proximate to the first edge of the main body 102 and the sample tube 126 may be coupled proximate to the second edge of the main body 102, as depicted in FIG. 3.

In various examples, the medical drain bag 100 may include at least one clamp 130 that is coupled to a corresponding tube of the plurality of tubes 120. For example, a first clamp 130 may be coupled to the input tube 122 and a second clamp 130 may be coupled to the sample tube 126, as depicted in FIGS. 2-4.

During use of the drain bag 100, liquid, such as PD effluent, may drain into the bag. The contents of the bag, which includes the PD effluent and may include other substances, such as contrast-enhancing agents, may be mixed manually or mechanically utilizing mixing devices, such as a rocker plate, a vibrating plate, or any other suitable device. Prior to or after the mixing, the patient 800 may detach their catheter from the input port 112, secure the first clamp 130 onto the input tube 122, and release the second clamp 130 from the sample tube 126, which may allow the liquid to flow from the reservoir 104 and into the imaging slide 300 for subsequent analysis.

Referring now to FIGS. 5A and 5B, views of an imaging slide 300 are provided. The imaging slide 300 may have a main body 304 that is pill-shaped and may have two straight edges 306 that are parallel to each other. The main body 304 may be flat such that a thickness of the main body 304 is less than 0.2 inch. The imaging slide 300 can comprise a medical-grade, optically clear, or substantially optically clear, material, such as a plastic or glass. The imaging slide 300 may be optimized for use with a digital microscope.

In various examples, the imaging slide 300 is configured to receive a flow of a liquid, such as a PD effluent, from a reservoir 104 of a medical drain bag 100. For example, the imaging slide 300 may include a channel 308 that is configured to receive the flow of the liquid. The channel 308 may be defined within and may be encased, at least partially, by the main body 304 of the imaging slide 300.

In various examples, the imaging slide 300 includes an imaging window 309 that at least partially defines the channel 308. The imaging window 309 can be at a location that an imaging system 500 images the liquid that is within the channel 308. The imaging window 309 can be optically clear, or substantially optically clear, such that light can pass through the imaging window 309 while minimizing excess absorption or scattering of the light from the imaging window materials. For example, optical clarity can be defined per testing methods defined in ASTM D-1003. In various examples, the imaging window 309 can comprise glass or a polymer. The imaging slide 300 can include two imaging windows 309 that are positioned on opposite sides of the imaging slide 300 so that light can pass through the imaging slide 300.

In various examples, and as discussed, the imaging slide 300 includes an inlet 302. The inlet 302 may be configured to allow a catheter, a sample port 116, or a sample tube 126 of a medical drain bag 100 to be coupled to the imaging slide 300. The inlet 302 may include threads to couple with the catheter, the sample port 116, or the sample tube 126 or may be configured to be friction fit with the catheter, the sample port 116, or the sample tube 126. The inlet 302 may be positioned upstream from the channel 308.

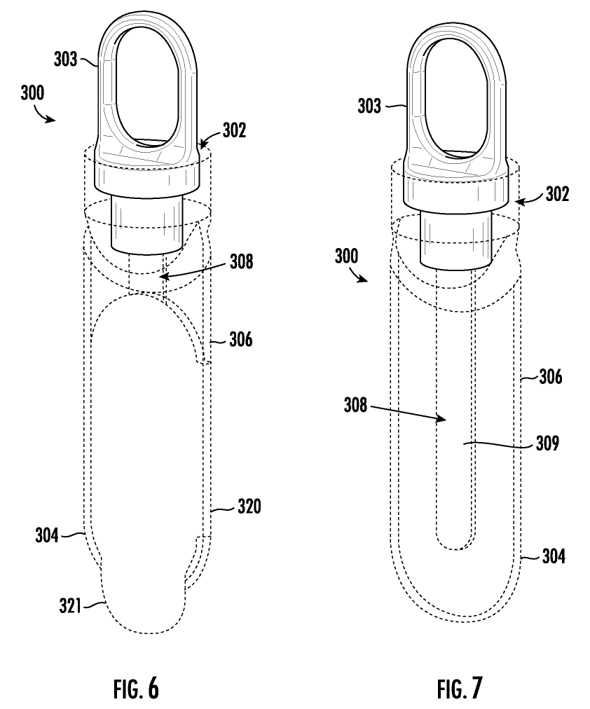

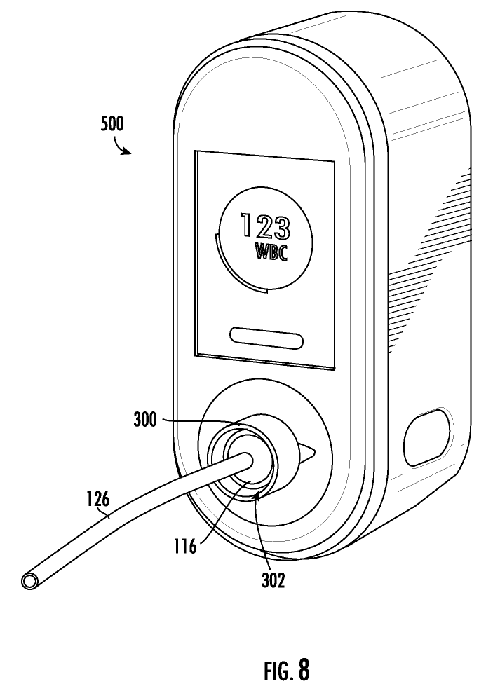

Referring now to FIGS. 6 and 7, views of an imaging slide 300 are provided, in accordance with an example embodiment. The imaging slide 300 of FIGS. 6 and 7 can be configured similarly to, or the same as, the imaging slide 300 of FIGS. 5A and 5B except that the imaging slide 300 may include a protective film 320 (as depicted in FIG. 6) that may be removed (as depicted in FIG. 7).

In various examples, the protective film 320 may be placed over the imaging window 309 of the imaging slide 300. For example, when the imaging slide 300 includes two imaging windows 309 that are positioned on opposite sides of the imaging slide 300, the protective film 320 may be placed over both the imaging windows 309. For example, the protective film 320 may wrap around an edge 306 of the main body 304 of the imaging slide 300 such that the protective film 320 is placed over both of the opposing sides of the imaging slide 300. In various example, the imaging slide 300 may include two separate protective films 320, each placed over a different imaging window 309, the imaging windows being on opposing sides of the imaging slide 300.

In various examples, the protective film 320 includes a tab 321. The tab 321 may extend beyond a periphery of a main body 304 of the imaging slide 300. For example, and as depicted in FIG. 6, the tab 321 may extend beyond a bottom edge of the main body 304 of the imaging slide 300. The tab 321 may facilitate removal of the protective film 320. The protective film 320 may protect the imaging window 309 from being scratched, or otherwise damaged, or from becoming dirty with, for example, smudges from the imaging slide 300 being handled. However, the protective film 320 may need to be removed prior to being imaged by an imaging system 500. As such, a user, such as a patient 800, may remove the protective film 320 by pulling on the tab 321 and removing the protective film 320 from the main body 304 of the imaging slide 300.

Referring now to FIG. 8, a view of an imaging system 500 is provided, in accordance with an example embodiment. The imaging system 500 may be configured to image imaging slide 300. The imaging system 500 may be configured to receive a main body 304 of the imaging slide 300. As depicted, the imaging system 500 may allow for the imaging slide 300 to be inserted into an orifice of the imaging system 500 while the imaging slide 300 is still connected to a sample port 116 of a medical drain bag 100.

In various examples, the safety cap 303 may be placed into the inlet 302 of the imaging slide 300 after removing the sample port 116 of the medical drain bag 100. Subsequently, the imaging slide 300 that includes the safety cap 303 may be placed into the orifice of the imaging system 500.

In various examples, the imaging system 500 comprises an illumination device and an imaging device. The imaging system 500 may be configured to receive the imaging slide 300 between the illumination device and the imaging device for imaging. The imaging system 500 may implement various imaging techniques, such as optical microscopy, ultraviolet fluorescence, digital holography, etc. The images captured by the imaging system 500 can be analyzed to count, identify, and/or classify particles and/or cells in the liquid, such as the PD effluent. As will be appreciated, certain types or quantities of particles or cells, such as white blood cells, may indicate that the patient 800 may have, or may be developing, an infection.

The medical drain bag 100 and the imaging slide 300 of the present disclosure have various benefits. For example, the medical drain bag 100 allows for the imaging slide 300 to be directly coupled to the medical drain bag 100, which simplifies sample collection. The imaging slide 300 may be previously coupled to the medical drain bag 100 such that an end user, such as a patient 800, does not need to couple the imaging slide to the drain bag 100 after receiving the medical drain bag 100 and/or prior to undergoing a procedure, such as a peritoneal dialysis procedure, to drain a liquid from their body, which simplifies the process. The imaging slide 300 may be placed directly within an imaging system 500 without being decoupled from the medical drain bag 100, which simplifies sample analysis and may prevent contamination of the sample within the imaging slide.

### Conclusion

The above descriptions of various embodiments of the subject disclosure and corresponding figures and what is described in the Abstract, are described herein for illustrative purposes, and are not intended to be exhaustive or to limit the disclosed embodiments to the precise forms disclosed. It is to be understood that one of ordinary skill in the art may recognize that other embodiments having modifications, permutations, combinations, and additions can be implemented for performing the same, similar, alternative, or substitute functions of the disclosed subject matter, and are therefore considered within the scope of this disclosure. Therefore, the disclosed subject matter should not be limited to any single embodiment described herein, but rather should be construed in breadth and scope in accordance with the appended claims below. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A medical drain bag comprising:
a main body that defines a reservoir, the main body configured to hold a liquid within the reservoir; and
a plurality of ports that are each in fluid communication with the reservoir, wherein the plurality of ports comprise:
an input port configured to be coupled to a catheter and receive the liquid through the catheter;
a syringe port that is configured to allow the liquid to flow from the reservoir and to a syringe; and
a sample port that is configured to allow the liquid to flow from the reservoir and to an imaging slide.

2. The medical drain bag of claim 1, wherein the medical drain bag comprises a plurality of tubes that are each coupled to the main body, wherein the plurality of tubes comprise:
an input tube that is coupled to the input port;
a syringe tube that is coupled to the syringe port; and
a sample tube that is coupled to the sample port.

3. The medical drain bag of claim 2, wherein the main body has a rounded rectangle shape that defines four edges, and wherein each of the plurality of tubes are coupled proximate to the same edge of the main body.

4. The medical drain bag of claim 2, wherein:
the main body has a rounded rectangle shape that defines a first edge that is opposite to a second edge,
the input tube and the syringe tube are coupled proximate to the first edge of the main body, and
the sample tube is coupled proximate to the second edge of the main body.

5. The medical drain bag of claim 2, further comprising a safety clamp coupled to the input tube, the syringe tube, or the sample tube.

6. The medical drain bag of claim 1, wherein the liquid is a peritoneal dialysis effluent.

7. An imaging slide configured to receive a flow of a liquid, the imaging slide comprising:
a channel configured to receive the flow of the liquid;
an imaging window that at least partially defines the channel; and
an inlet, wherein the inlet is configured to allow a catheter or a sample tube of a medical drain bag to be coupled to the imaging slide.

8. The imaging slide of claim 7, wherein the imaging window is optically clear, wherein the imaging slide is configured to be imaged by an imaging system, wherein the imaging system comprises an illumination device and an imaging device, and wherein the imaging system is configured to receive the imaging slide between the illumination device and the imaging device.

9. The imaging slide of claim 7, further comprising a protective film that is placed over the imaging window.

10. The imaging slide of claim 9, wherein the protective film comprises a tab that extends beyond a periphery of a main body of the imaging slide.

11. The imaging slide of claim 9, wherein the protective film wraps around an edge of a main body of the imaging slide such that the protective film is placed over opposing sides of the imaging slide.

12. A medical drain bag comprising:
a main body that defines a reservoir, the main body configured to hold a liquid within the reservoir; and
a plurality of ports that are each in fluid communication with the reservoir, wherein the plurality of ports comprise:
an input port configured to be coupled to a catheter and receive the liquid through the catheter;
a syringe port that is configured to allow the liquid to flow from the reservoir and to a syringe; and
a sample port that is coupled to an imaging slide and is configured to allow the liquid to flow from the reservoir and to the imaging slide,
wherein the imaging slide comprises:
a channel configured to receive the liquid;
an imaging window that at least partially defines the channel; and
an inlet, wherein the inlet is coupled to the sample port.

13. The medical drain bag of claim 12, wherein the medical drain bag comprises a plurality of tubes that are each coupled to the main body, wherein the plurality of tubes comprise:
an input tube that is coupled to the input port;
a syringe tube that is coupled to the syringe port; and
a sample tube that is coupled to the sample port.

14. The medical drain bag of claim 13, wherein the main body has a rounded rectangle shape that defines four edges, and wherein each of the plurality of tubes are coupled proximate to the same edge of the main body.

15. The medical drain bag of claim 13, wherein:
the main body has a rounded rectangle shape that defines a first edge that is opposite to a second edge,
the input tube and the syringe tube are coupled proximate to the first edge of the main body, and
the sample tube is coupled proximate to the second edge of the main body.

16. The medical drain bag of claim 13, further comprising a safety clamp coupled to the input tube, the syringe tube, or the sample tube.

17. The medical drain bag of claim 12, wherein the imaging window is optically clear, wherein the imaging slide is configured to be imaged by an imaging system, wherein the imaging system comprises an illumination device and an imaging device, and wherein the imaging system is configured to receive the imaging slide between the illumination device and the imaging device.

18. The medical drain bag of claim 12, further comprising a protective film that is placed over the imaging window.

19. The medical drain bag of claim 18, wherein the protective film comprises a tab that extends beyond a periphery of a main body of the imaging slide.

20. The medical drain bag of claim 18, wherein the protective film wraps around an edge of a main body of the imaging slide such that the protective film is placed over opposing sides of the imaging slide.
